(19) United States
(12) Patent Application Publication
(10) Pub. No.: US 2020/0261681 A1
(43) Pub. Date: Aug. 20, 2020

(54) CUSTOM CONTOURED FRAME FOR PATIENT INTERFACE DEVICE

(71) Applicant: KONINKLIJKE PHILIPS N.V., EINDHOVEN (NL)

(72) Inventor: PETER CHI FAI HO, EINDHOVEN (NL)

(21) Appl. No.: 16/063,716

(22) PCT Filed: Dec. 20, 2016
(86) PCT No.: PCT/IB2016/057803

§ 371 Date: Jun. 19, 2018

Related U.S. Application Data
(60) Provisional application No. 62/272,189, filed on Dec. 29, 2015.

(51) Int. Cl. A61M 16/06

(52) U.S. Cl. CPC A61M 16/0622; B33Y 80/00; A61M 16/0633

(57) ABSTRACT

A frame for use in a patient interface device for delivering a flow of breathing gas to the airway of a patient includes a first end structured to be disposed at or about the forehead of the patient and a second end opposite the first end. The second end is structured to be coupled to a adapted to sealingly engage the face of the patient about an airway of the patient. The frame further includes a spine extending between the first end and the second end. The spine is sized and configured to follow the profile of the patient along at least a portion of each of the forehead, brow and nose of the patient.

### CROSS-REFERENCE TO RELATED APPLICATIONS

This patent application claims the priority benefit under 35 U.S.C. § 119(e) of U.S. Provisional Application No. 62/272,189 filed on Dec. 29, 2015, the contents of which are herein incorporated by reference.

### BACKGROUND OF THE INVENTION

### 1. Field of the Invention

The present invention pertains to systems for delivering a flow of breathing gas to the airway of a patient. The present invention also pertains to interface devices for use in delivering a flow of breathing gas to the airway of a patient. The present invention further pertains to frames for use in such patient interface devices.

### 2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is OSA. Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device including a mask component having a soft, flexible cushion on the face of a patient. The mask component may be, without limitation, a nasal mask that covers the patient's nose, a nasal cushion having nasal prongs that are received within the patient's nares, a nasal/oral mask that covers the patient's nose and mouth, or a full face mask that covers the patient's face. Such patient interface devices may also employ other patient contacting components, such as forehead supports, cheek pads and chin pads. The patient interface device is connected to a gas delivery tube or conduit and interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

Because such masks are typically worn for an extended period of time, a variety of concerns must be taken into consideration. For example, in providing CPAP to treat OSA, the patient normally wears the patient interface device all night long while he or she sleeps. One concern in such a situation is that the patient interface device is properly fitted and thus as comfortable as possible; otherwise the patient may avoid wearing the interface device, thus defeating the purpose of the prescribed pressure support therapy. It is also important that the overall size of the interface device be minimized, so as to not be overly cumbersome on the face of the patient.

### SUMMARY OF THE INVENTION

Accordingly, it is an object of the present invention to provide an interface device that provides a comfortable, secure fit while minimizing obtrusiveness. This object is achieved according to one embodiment of the present invention by providing a frame for use in a patient interface device for delivering a flow of breathing gas to the airway of a patient. The frame comprises: a first end structured to be disposed at or about the forehead of the patient; a second end opposite the first end, the second end structured to be coupled to a cushion which is adapted to sealingly engage the face of the patient about an airway of the patient; and a spine extending between the first end and the second end, the spine being sized and configured to follow the profile of the patient along at least a portion of each of the forehead and nose of the patient.

The spine may include a plurality of retention members extending therefrom, each of the retention members being structured to engage and couple a main conduit to the frame.

The first end may comprise a first coupling conduit structured to be coupled to a delivery conduit of a system for delivering the flow of breathing gas to the patient. The frame may be formed as a first portion of a unitary member and the first coupling conduit may be integrally formed as a second portion of the unitary member.

The frame may include a main conduit extending between the first coupling conduit and the second end of the frame along the spine, the main conduit being structured to conduct the flow of breathing gas from the coupling member to the cushion. The frame may be formed as a first portion of a unitary member and the main conduit may be integrally formed as a second portion of the unitary member.

The second end may comprise a second coupling conduit structured to be coupled to a cushion which is adapted to sealingly engage about an airway of the patient. The second coupling conduit may comprise an exhalation device.

The frame may be formed via an additive manufacturing process.

The object is also achieved according to another embodiment of the present invention by providing a patient interface device for delivering a flow of breathing gas to the airway of a patient. The interface device comprises a frame which comprises: a first end structured to be disposed at or about the forehead of the patient; a second end opposite the first end; and a spine extending between the first end and the second end, the spine being sized and configured to follow the profile of the patient along at least a portion of each of the forehead and nose of the patient. The interface device also comprises: a cushion coupled to the second end of the spine, the cushion being adapted to sealingly engage the face of the patient about an airway of the patient; and a main conduit which extends between the first end and the second end of the frame, the conduit being structured to receive the flow of breathing gas and conduct the flow of breathing gas to the cushion.

The first end may comprise a first coupling conduit coupled to the main conduit and structured to be coupled to a delivery conduit of a system for delivering the flow of breathing gas to the patient. The frame may be formed as a first portion of a unitary member and the first coupling conduit may be formed as a second portion of the unitary member.

The second end may comprise a second coupling conduit coupled to the main conduit and to the cushion. The frame may be formed as a first portion of a unitary member and the main conduit may be formed as a second portion of the unitary member.

The first end may comprise a first coupling conduit coupled to the main conduit and structured to be coupled to a delivery conduit of a system for delivering the flow of breathing gas to the patient; the second end may comprise a second coupling conduit coupled to the main conduit and to the cushion; and the frame may be formed as a first portion of a unitary member with the first coupling conduit integrally formed as a second portion of the unitary member, and the second coupling conduit integrally formed as a third portion of the unitary member. The main conduit may be integrally formed as a fourth portion of the unitary member.

The second coupling conduit may comprise an exhalation device.

The frame may be formed via an additive manufacturing process.

The object is also achieved according to yet a further embodiment of the present invention by providing a system for delivering a flow of breathing gas to the airway of a patient. The system comprises: a pressure generating device which is structured to generate the flow of breathing gas; a delivery conduit coupled to the pressure generating device and structured to receive the flow of breathing gas from the pressure generating device; and a patient interface device. The patient interface device comprises a frame which comprises: a first end structured to be disposed at or about the forehead of the patient; a second end opposite the first end; and a spine extending between the first end and the second end, the spine being sized and configured to follow the profile of the patient along at least a portion of each of the forehead and nose of the patient. The patient interface device also comprises: a cushion coupled to the second end of the spine, the cushion being adapted to sealingly engage the face of the patient about an airway of the patient; and a main conduit extending between the first end and the second end of the frame, the conduit being structured to receive the flow of breathing gas from the deliver conduit and conduct the flow of breathing gas to the cushion.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a side isometric view of a patient interface device and a portion of a conduit shown connected to a gas flow/pressure generating system (shown schematically) to form a system adapted to provide a regiment of respiratory therapy to a patient according to one exemplary embodiment of the invention;

FIG. 2 is a side view of the patient interface device of FIG. 1 shown disposed on a portion of the face of a user according to one exemplary embodiment of the invention;

FIG. 3 is a side view of the frame of the patient interface device of FIG. 1;

FIG. 3B is a sectional view of a portion of the frame of FIG. 3 taken along line B-B;

FIG. 4 is a front view of the frame of the patient interface device of FIG. 1;

FIG. 5 is a rear view (patient facing side) of the frame of the patient interface device of FIG. 1;

FIG. 6 is a bottom view of the frame of the patient interface device of FIG. 1 also showing a portion of the rear (patient facing side) of the frame;

FIG. 7 is a top view of the frame of the patient interface device of FIG. 1;

FIG. 8 is a bottom view of the frame of the patient interface device of FIG. 1 also showing a portion of the front of the frame;

FIG. 9 is a cross-section view of the patient interface device of FIG. 1;

FIG. 10 is an isometric side view of a patient interface device according to one exemplary embodiment of the invention;

FIG. 11 is a cross-sectional view of the patient interface device of FIG. 10; and

FIGS. 12-15 are side views of patient interface devices according to exemplary embodiments of the invention shown disposed on the head of a patient. Such interface devices are also shown coupled to a conduit shown connected to a gas flow/pressure generating system (shown schematically) to form a system adapted to provide a regiment of respiratory therapy to a patient according to exemplary embodiments of the invention.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

As used herein, the word “patient” or “user” shall be used interchangeably to refer to the person to which the interface device is delivering a flow of breathing gas.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

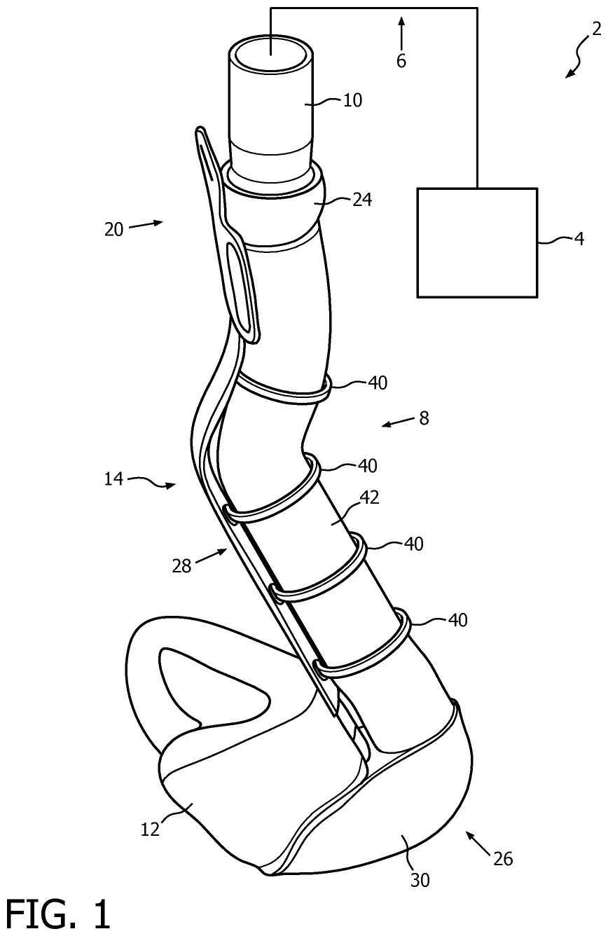

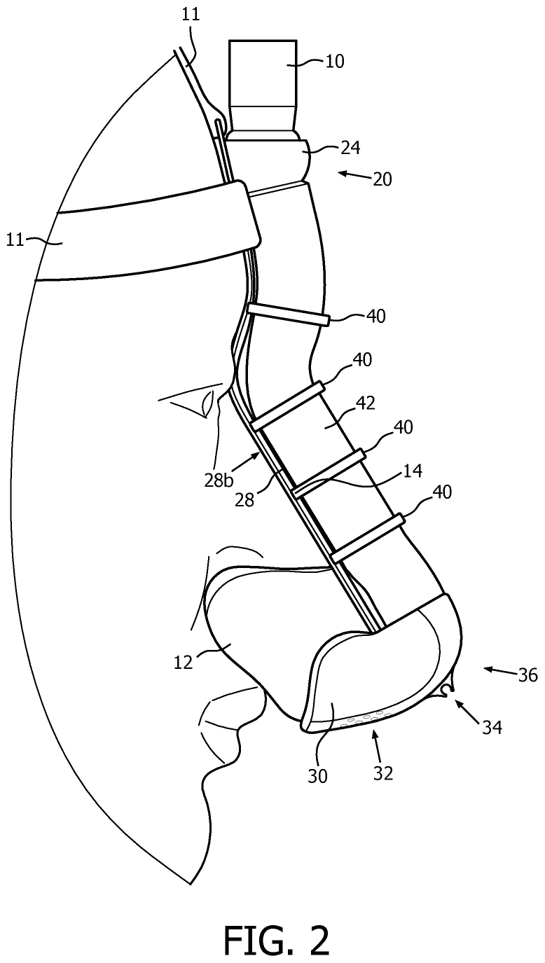

A system 2 adapted to provide a regimen of respiratory therapy to a patient according to one exemplary embodiment of the invention is generally shown in FIG. 1. System 2 includes a pressure generating device 4 (shown schematically), a delivery conduit 6 (shown partially schematically), and a patient interface device 8 having a fluid coupling conduit 10. FIG. 2 shows patient interface device 8 disposed on the face of an example user and generally secured thereto via a strap or other suitable headgear 11. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 8 through fluid coupling conduit 10, which in the illustrated embodiment is an elbow connector. Delivery conduit 6 and user interface device 8 are often collectively referred to as a patient circuit.

A BiPAP® device is a bi-level device in which the pressure provided to the patient varies with the patient's respiratory cycle, so that a higher pressure is delivered during inspiration than during expiration. An auto-titration pressure support system is a system in which the pressure varies with the condition of the patient, such as whether the patient is snoring or experiencing an apnea or hypopnea. For present purposes, pressure/flow generating device 4 is also referred to as a gas flow generating device, because flow results when a pressure gradient is generated. The present invention contemplates that pressure/flow generating device 4 is any conventional system for delivering a flow of gas to an airway of a patient or for elevating a pressure of gas at an airway of the patient, including the pressure support systems summarized above and non-invasive ventilation systems.

In the exemplary embodiment illustrated in FIGS. 1 and 2, patient interface device 8 is depicted as a nasal mask which includes a user sealing assembly or cushion 12 coupled to a generally rigid frame 14. However, it is to be appreciated that other types of cushions, such as, without limitation, a nasal pillows cushion or a full face cushion, which facilitates the delivery of the flow of breathing gas to the airway of a user, may be generally substituted for cushion 12 while remaining within the scope of the present invention. In the exemplary embodiment illustrated in FIGS. 1 and 2, patient interface device 8 is depicted as being coupled to conduit 6 via fluid coupling conduit 10 which is in the form of a male ball joint swivel coupling. However, it is to be appreciated that conduit 6 may be coupled to patient interface device 8 via any suitable coupling or alternatively may be directly coupled to patient interface device 8 without the use of any intermediary coupling without varying from the scope of the present invention.

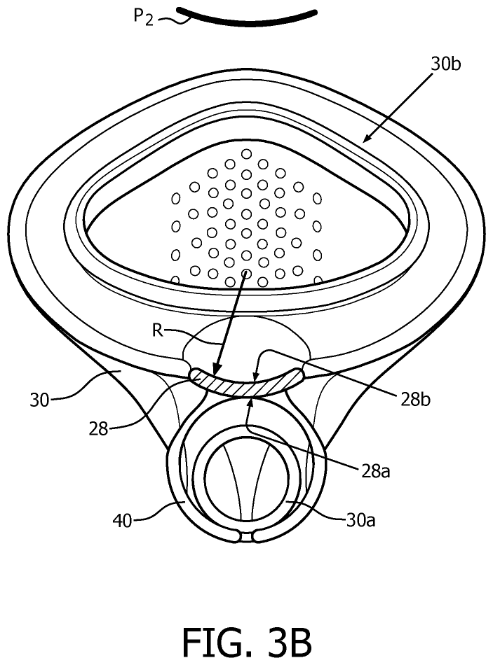

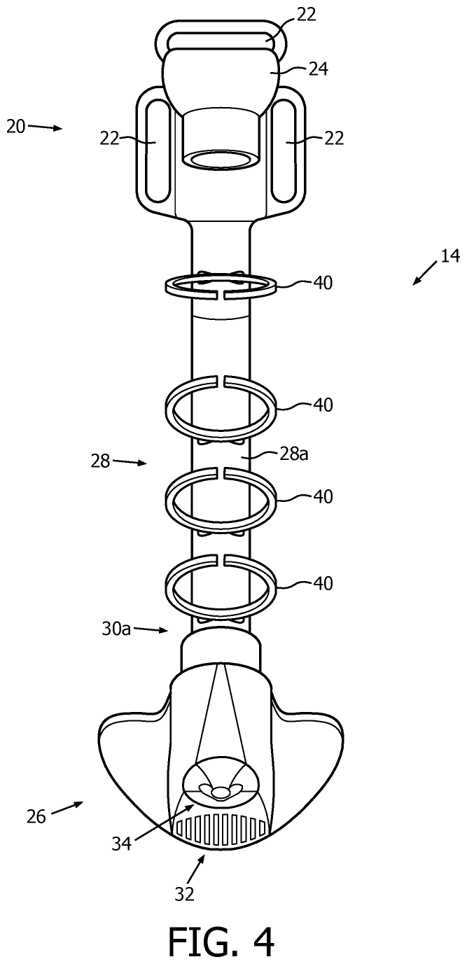

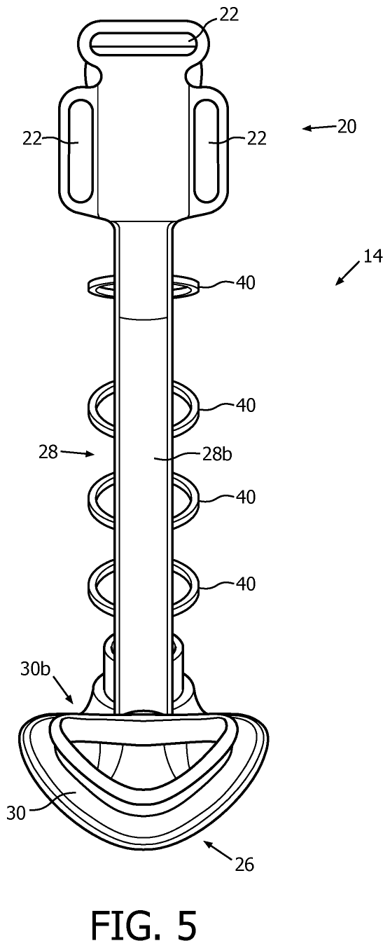

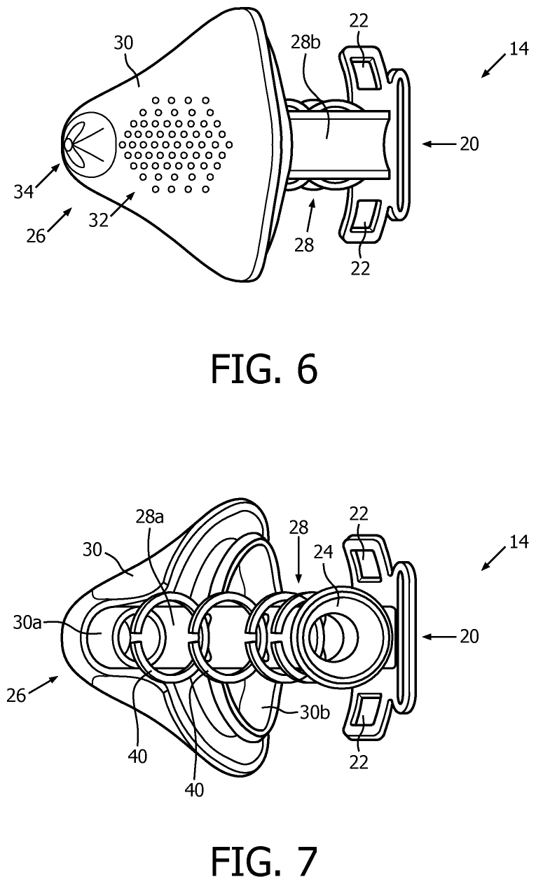

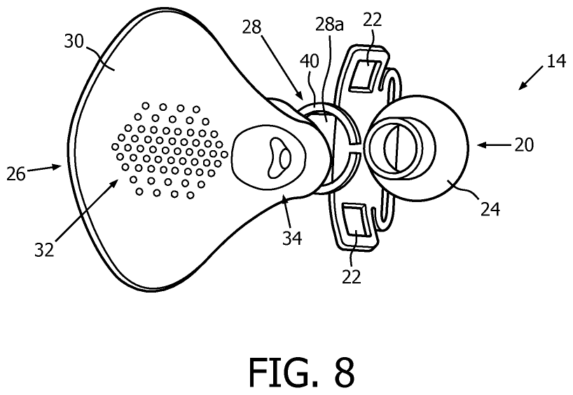

Referring to FIGS. 2-8, frame 14 includes a first end 20 structured to be disposed at or about the forehead of the patient (FIG. 2). In order to assist in securing frame 14 to a patient, first end 20 may include one or more slots 22 defined therein or other suitable structures for engaging with a headgear (e.g., without limitation, headgear 11) or other suitable mechanism. In the exemplary embodiment illustrated in FIGS. 1-8, first end 20 includes a first coupling conduit 24 which is structured to be coupled to delivery conduit 6 of system 2 either directly or indirectly via an intermediate member (e.g., without limitation, fluid coupling conduit 10). In the exemplary embodiment illustrated in FIGS. 1-8, first coupling conduit 24 is in the form of a female ball joint swivel coupling. It is to be appreciated, however, that first coupling conduit 24 may comprise any suitable coupling member without varying from the scope of the present invention.

Frame 14 further includes a second end 26 disposed opposite the first end and a spine 28 extending between first end 20 and second end 26. Second end 26 is structured to be coupled to a cushion, such as cushion 12 of FIGS. 1 and 2, which is adapted to sealingly engage an airway of the patient. In the example embodiment illustrated in FIGS. 1-8, second end 26 includes a second coupling conduit 30 which has an area which generally increases from a first end 30a to a second end 30b, which is structured to have cushion 12 sealingly engaged thereto. Second coupling conduit 30 may include an integrated exhalation device 32 which, in the exemplary embodiment illustrated in FIGS. 1-8 is a plurality of exhaust ports. It is to be appreciated, however, that other suitable arrangements may be employed without varying from the scope of the present invention. Second coupling conduit 30 may further include a mount 34 or other suitable structure which may be used to further secure interface device 8 to the head of a user.

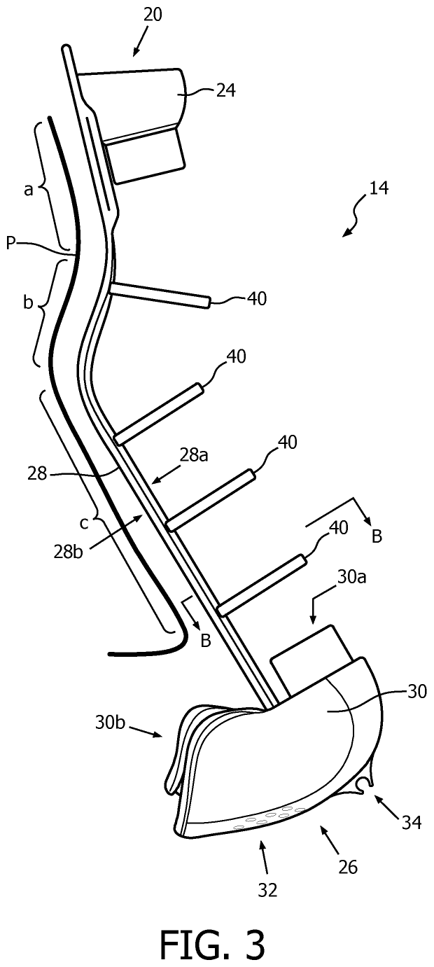

As shown in FIGS. 2 and 3, spine 28 is sized and configured to follow the profile P (FIG. 3) of the patient along at least a portion of the forehead, brow and nose of the patient (profile P is shown pulled away from spine 28 in FIG. 3 for exemplary purposes). More particularly, as shown in FIGS. 2 and 3, spine 28 includes a first side 28a which faces away from the patient and a second side 28b which faces toward the patient. Additionally, since spine 28 is sized and configured to follow the profile of the patient along at least a portion of the forehead, brow and nose of the patient, second side 28b of spine 28 is generally disposed against at least portions of the forehead, brow and nose of the patient. It is to be appreciated that profile P of a particular patient may be determined in any suitable manner without varying from the scope of the present invention. For example, a 3D scan of the patient's face may be used. In a more simplistic approach, a few selected landmarks (points) selected along the patients forehead, brow and nose may be used to determine the profile P. As shown in the exemplary embodiment illustrated in FIG. 3, a typical profile P may have three rather distinct generally linear segments a, b, c. It is to be appreciated, however, that facial profiles having other quantities of segments may be employed without varying from the scope of the present invention.

As shown in the sectional view of FIG. 3B, spine 28 may also have a curved profile so as to also generally follow the profile P₂ of the patient, wherein profile P₂ is perpendicular to profile P. In the example shown in FIG. 3B profile P₂ is generally defined by a radius R. It is to be appreciated that such arrangement is given for example purposes only and that other shapes may be utilized without varying from the scope of the present invention. It is also to be appreciated that such arrangement of spine 28 provides for a frame which may readily be secured to a patient in a manner which is more comfortable and secure than existing solutions.

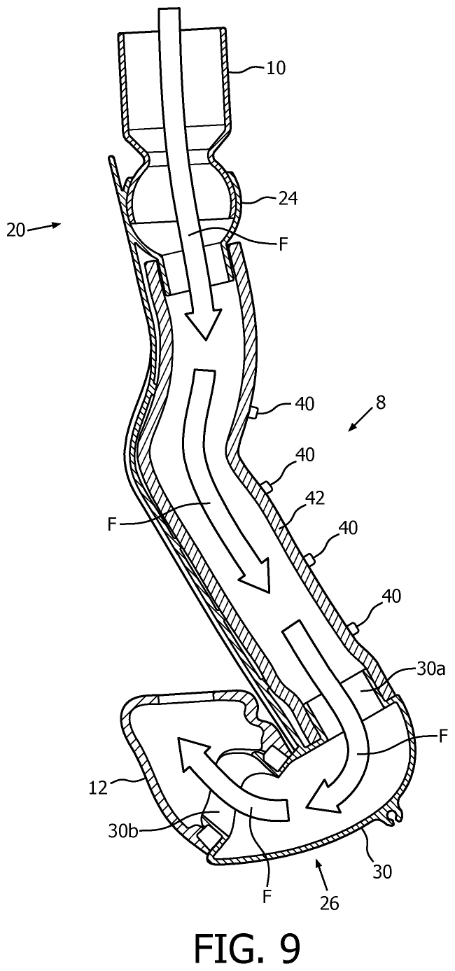

Spine 28 includes a plurality of retention members 40 extending therefrom. More particularly, each retention member 40 extends from first side 28a so as to extend generally away from the user. Each retention member 40 is structured to engage and couple a main conduit 42, which extends between first coupling conduit 24 and second coupling conduit 30. Each retention member 40 may extend completely around, or about only a portion of the circumference of main conduit 42 in order to couple main conduit 42 to frame 14. Referring to FIG. 1 and the cross-sectional view of FIG. 9, it is thus to be appreciated from the foregoing description that when in use, a flow (shown by arrows F in FIG. 9) of breathing gas produced by pressure generating device 4 received at first coupling conduit 24 passes therethrough and into main conduit 42. Flow F then passes through main conduit 42 and into second coupling 30. Flow F then passes through second coupling conduit and into cushion 12, before finally entering the airway of the patient.

In exemplary embodiments of the present invention frame 14 has been formed using an additive manufacturing processes such as DMP (Direct Metal Printing) and DMLS (Direct Metal Laser Sintering). Using such processes frames 14 have been formed from Titanium, stainless steel and aluminum alloys. Additionally, polymers may be employed using a SLS (Selective Laser Sintering) process. It is to be appreciated that other processes and materials may be employed without varying from the scope of the present invention. By utilizing an additive manufacturing process, one or more of first coupling conduit 24, second coupling conduit 30, retention members 40, and/or main conduit 42 may be formed as integral portions of frame 14. For example, in the exemplary embodiment shown in the cross-sectional view of FIG. 9, first coupling conduit 24, second coupling conduit 30 and spin 28 are all formed as integral portions of the same member (e.g., without limitation, from Titanium, stainless steel, aluminum alloy, polymer). In contrast, main conduit 42 was sourced from a silicone tube or any bendable rubber, e.g., medical tube materials (i.e., PVC (Vinyl), TPU (Thermoplastic polyurethane), TPE (thermoplastic elastomer), PEBA (Polyether Block Amide)) which was cut to length to match spine 24 (and be housed by retention members 40) and then installed in interface device 8. Alternatively, first coupling conduit 24 and second coupling conduit 30 could be common components (i.e., having the same size and structure and dimensions for use with any spine 28) which are prefabricated via injection molding or other suitable process and then bonded to a particular spine 28.

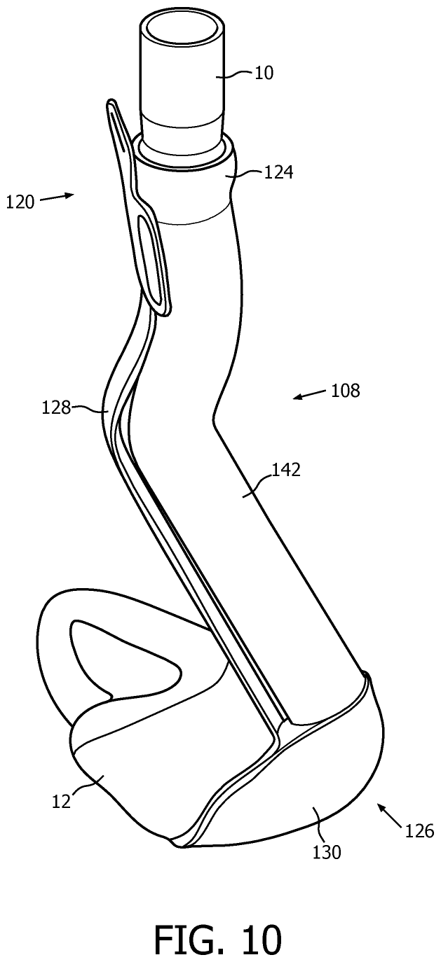

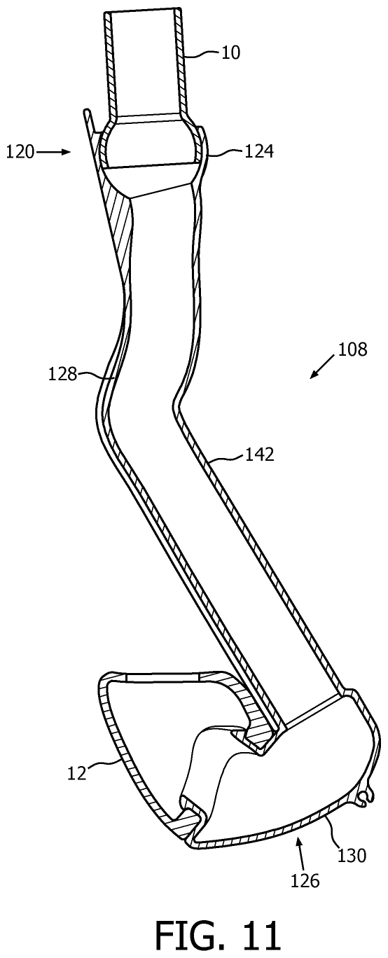

As another example, FIGS. 10 and 11 illustrate a patient interface device 108 which is of a generally similar arrangement as patient interface 8 (previously described), except patient interface 108 includes a main conduit 142 which is integrally formed with a spine 128 as well as a first coupling conduit 124 and a second coupling conduit 130. Once again, it is to be appreciated that the arrangement shown in FIGS. 10 and 11 is made possible by using an additive manufacturing process such as previously described.

As an alternative to individually customizing a spine 28 for a particular patient, a semi-custom spine 28 can be generated from careful clustering of specific groups of faces or scans to create average faces. From such average faces an average facial profile can be created to which spines may be tailored to fit a group of individuals with similar facial structure. For example, groups can be clustered by genders, body builds and ethnicities. The number of clusters will depend on how specific are the groups being considered.

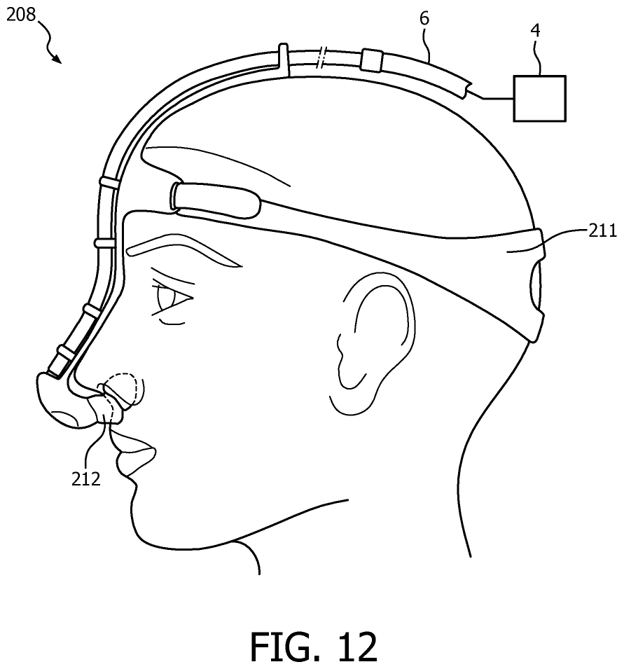

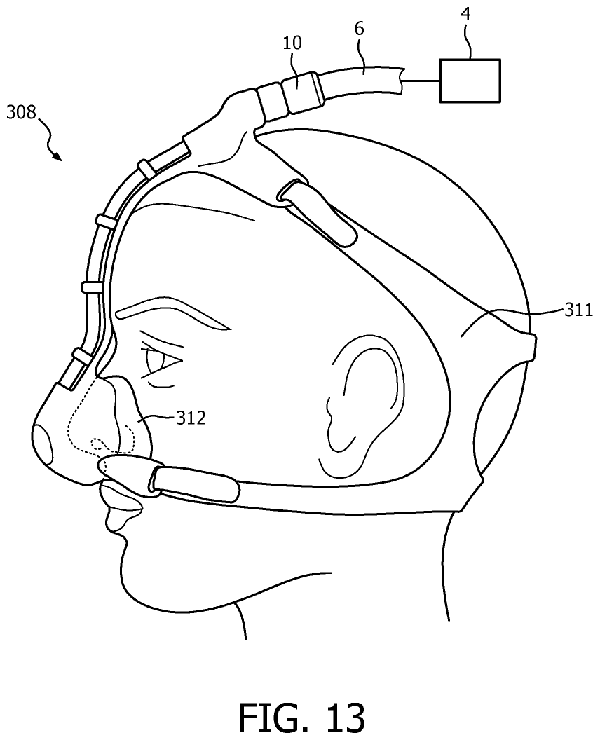

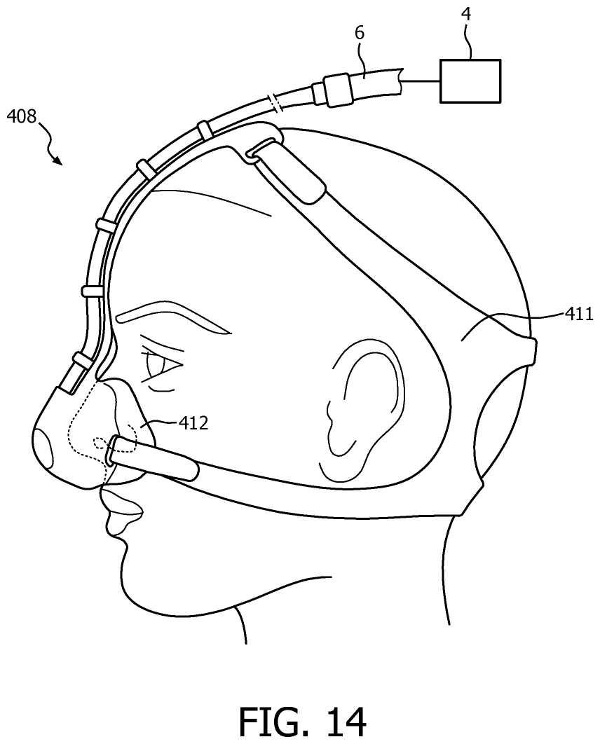

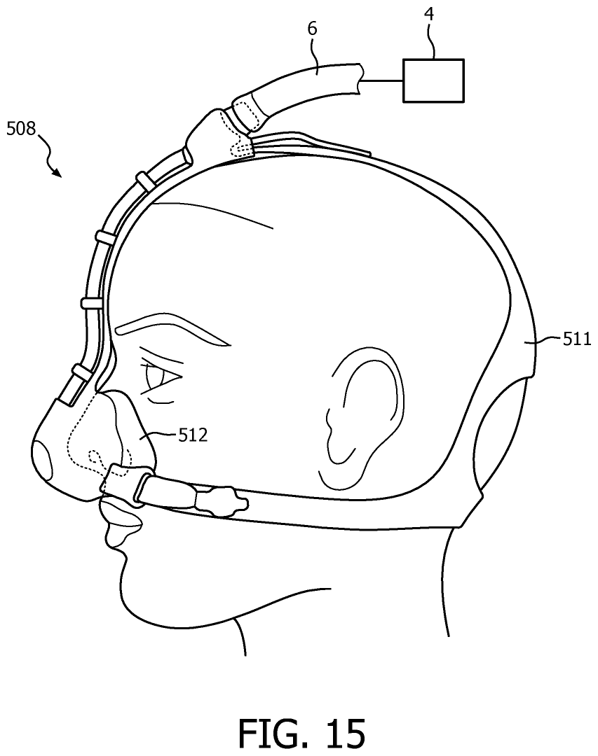

FIGS. 12-15 show additional example embodiments of patient interface devices 208, 308, 408 and 508 shown coupled on the head of a patient via example headgear 211, 311, 411, 511 and coupled to pressure generating device 4 via conduit 6 (as previously discussed in regard to FIG. 1). FIG. 12 shows an example embodiment employing individual nasal cushions 212, while FIGS. 13-15 show example embodiments which employ a nasal cushion 312, 412 and 512. From such additional examples, as well as from the previously described embodiments, it is to be appreciated that various details of the invention (e.g., without limitation, patient sealing portion, length/diameter of tubing, size/type of headgear, etc.) may be varied without varying from the scope of the present invention.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A frame for use in a patient interface device for delivering a flow of breathing gas to the airway of a patient, the frame comprising:
a first end structured to be disposed at or about the forehead of the patient;
a second end opposite the first end, the second end structured to be coupled to a cushion which is adapted to sealingly engage the face of the patient about an airway of the patient; and
a spine extending between the first end and the second end, the spine being sized and configured to be disposed against and to follow the profile of the patient along at least a portion of each of the forehead and nose of the patient.

2. The frame of claim 1, wherein the spine includes a plurality of retention members extending therefrom, each of the retention members being structured to engage and couple a main conduit to the frame.

3. The frame of claim 1, wherein the first end comprises a first coupling conduit structured to be coupled to a deliver conduit of a system for delivering the flow of breathing gas to the patient, and wherein the frame is formed as a first portion of a unitary member and wherein the first coupling conduit is integrally formed as a second portion of the unitary member.

4. (canceled)

5. The frame of claim 3, further comprising a main conduit extending between the first coupling conduit and the second end of the frame along the spine, the main conduit being structured to conduct the flow of breathing gas from the coupling member to the cushion, and wherein the frame is formed as a first portion of a unitary member and wherein the main conduit is integrally formed as a second portion of the unitary member.

6. (canceled)

7. The frame of claim 1, wherein the second end comprises a second coupling conduit structured to be coupled to a cushion which is adapted to sealingly engage about an airway of the patient.

8. The frame of claim 7, wherein the second coupling conduit comprises an exhalation device.

9. The frame of claim 1, wherein the frame is formed via an additive manufacturing process.

10. A patient interface device for delivering a flow of breathing gas to the airway of a patient, the interface device comprising:
a frame comprising:
a first end structured to be disposed at or about the forehead of the patient,
a second end opposite the first end, and
a spine extending between the first end and the second end, the spine being sized and configured to be disposed against and to follow the profile of the patient along at least a portion of each of the forehead and nose of the patient;
a cushion coupled to the second end of the spine, the cushion being adapted to sealingly engage the face of the patient about an airway of the patient; and
a main conduit extending between the first end and the second end of the frame, the conduit being structured to receive the flow of breathing gas and conduct the flow of breathing gas to the cushion.

11. The patient interface device of claim 10, wherein the first end comprises a first coupling conduit coupled to the main conduit and structured to be coupled to a delivery conduit of a system for delivering the flow of breathing gas to the patient and wherein the frame is formed as a first portion of a unitary member and wherein the first coupling conduit is formed as a second portion of the unitary member.

12. (canceled)

13. The patient interface device of claim 10, wherein the second end comprises a second coupling conduit coupled to the main conduit and to the cushion, and wherein the frame is formed as a first portion of a unitary member and wherein the second coupling conduit is formed as a second portion of the unitary member.

14. (canceled)

15. The patient interface device of claim 10, wherein:
the first end comprises a first coupling conduit coupled to the main conduit and structured to be coupled to a delivery conduit of a system for delivering the flow of breathing gas to the patient;
the second end comprises a second coupling conduit coupled to the main conduit and to the cushion; and
the frame is formed as a first portion of a unitary member, the first coupling conduit is integrally formed as a second portion of the unitary member, and the second coupling conduit is integrally formed as a third portion of the unitary member.

16. The patient interface device of claim 15, wherein the main conduit is integrally formed as a fourth portion of the unitary member.

17. The patient interface device of claim 13, wherein the second coupling conduit comprises an exhalation device.

18. The patient interface device of claim 10, wherein the frame is formed via an additive manufacturing process.

19. A system for delivering a flow of breathing gas to the airway of a patient, the system comprising:
(a) a pressure generating device which is structured to generate the flow of breathing gas;
(b) a delivery conduit coupled to the pressure generating device and structured to receive the flow of breathing gas from the pressure generating device; and
(c) a patient interface device comprising:
(1) a frame comprising:
(i) a first end structured to be disposed at or about the forehead of the patient,
(ii) a second end opposite the first end, and
(iii) a spine extending between the first end and the second end, the spine being sized and configured to be disposed against and to follow the profile of the patient along at least a portion of each of the forehead and nose of the patient;
a cushion coupled to the second end of the spine, the cushion being adapted to sealingly engage the face of the patient about an airway of the patient; and
a main conduit extending between the first end and the second end of the frame, the conduit being structured to receive the flow of breathing gas from the deliver conduit and conduct the flow of breathing gas to the cushion.
